(19) United States
(12) Patent Application Publication
(10) Pub. No.: US 2026/0237526 A1
(43) Pub. Date: Aug. 13, 2026

(54) PATIENT EXAMINATION AND EDUCATION SYSTEM

(71) Applicant: Anthony R. Cucci, Akron, OH (US)

(72) Inventor: Anthony R. Cucci, Akron, OH (US)

(21) Appl. No.: 19/047,706

(22) Filed: Feb. 7, 2025

(51) Int. Cl. G16H 70/20; A61G 7/05; G16H 80/00

(52) U.S. Cl. CPC G16H 70/20; A61G 7/0502; G16H 80/00

(57) ABSTRACT

A patient examination and education system employs cover sheets for an articulating medical examination table as a source of information and a focus point for dialogue between physician and patient. A roll of sanitary cover sheets is maintained on a rear surface of the back of an examination table. The examination table sheets bear medical information and accommodate instruction and dialogue between doctor and patient regarding medical conditions and associated treatments for the patient. The sheets not only facilitate, but also prompt discussion between physician and patient and provide medical illustrations to aid in such discussions. Notes may be written upon the sheets and the sheets separated from the roll and taken with the patient after the examination session. The illustrations are keyed to particular specialties or practices of the physician, surgeon or other practitioner.

### TECHNICAL FIELD

The invention herein resides in the art of medical services and, more particularly, to the services of medical examinations to diagnose physical conditions of patients and their related progress in responding to treatment. More specifically, the invention relates to a medical patient examination and education system that accommodates a visual and verbal dialogue between and among patients, physicians, and others of interest to enable a mutual understanding of a physical diagnosis, options for treatment and treatment effectiveness.

### BACKGROUND OF THE INVENTION

The interaction between physicians and patients during outpatient office visits has changed little over the history of medicine. This is despite the increasing complexity of diseases, procedures, surgeries and treatment. The time spent between physicians and patients has continued to decrease due to increasing pressure for physicians to see more patients in a given period of time. Moreover, communication between physicians and patients is often in medical terms that patients do not understand. The combination of these factors has resulted in patients often leaving a doctor's visit unsatisfied, confused, and not fully understanding issues involving their own health and an associated plan of care.

Rarely, physicians may use visualization techniques, such as drawing on a notepad or the like, but these tools and approaches are often (1) too limited in space to allow the physician to fully demonstrate or draw out the complexities of the patient's health, (2) too small to allow for appropriate physician-patient interaction (or such interaction with accompanying family members), and most frequently (3) not even available in the examination room. Additionally, it is tedious and time-consuming for the physician to continue to draw the same basic images repeatedly. The time consumed could be better spent communicating directly with the patient or accompanying family members. Overall, the use of notepads and other small visual aids are outdated inadequate means of communication. The techniques for patient involvement must change, because medicine is changing, as is the patient-doctor experience.

Currently, physicians rarely use visualization as part of discussions/education with patients and family due to a lack of time and also a lack of visual aids. There remains a large communication gap between physician and patient, resulting in significant levels of patient misunderstanding due to medical terminology and a lack of visualization presented by the physician. This not only reduces patient satisfaction and an understanding of the patient's own health, but it also adds to increasing anxiety in the medical treatment experience.

### DISCLOSURE OF INVENTION

In light of the foregoing, it is a first aspect of the invention to provide a patient examination and education system that bridges the gap between physician communication and patient understanding.

Another aspect of the invention is the provision of a patient examination and education system that provides specialty-specific pre-printed images to facilitate physician communication and patient understanding.

Another aspect of the invention is the provision of a patient examination and education system that provides specialty-specific pre-printed images on dispensable and disposable examination table cover sheets.

Still a further aspect of the invention is the provision of a patient examination and education system that accepts manual markings that may be made by the physician or patient during physician-patient discussions.

Yet a further aspect of the invention is the provision of a patient examination and education system in which the disposable and dispensable cover sheet are readily dispensed from a roll and can be separated from the roll to be taken by the patient at the end of the physician-patient meeting.

Yet an additional aspect of the invention is the provision of a patient examination and education system wherein pre-printed images on rolls of cover sheet material are taken from a broad range of medical specialties.

Still an additional aspect of the invention is the provision of a patient examination and education system that prompts discussion and focuses attention of the physician and patient to the physical region of the patient's body that is of concern, making the examination and discussion more efficient.

Yet an additional aspect of the invention is the provision of a patient examination and education system that enhances the patient-physician interaction and the patient education and resulting experience.

Still a further aspect of the invention is the provision of a patient examination and education system that is conducive to implementation with a broad range of medical specialties and is easily implemented with state-of-the-art examination table cover sheets and readily available physiological illustrations.

The foregoing and other aspects of the invention that will become apparent herein are achieved by a patient examination and education system, comprising: an examination table having a seat and a back in articulating interconnection, said seat having an upper surface for receiving and supporting a lower portion of a patient and said back having a front surface for supporting an upper portion of the patient; and a dispenser of information relevant to medical conditions of patients maintained on a rear surface of said back, said dispenser selectively dispensing said information over at least one of said front surface of said back and upper surface of said seat.

Other aspects of the invention that will become apparent herein are achieved by a method of a physician medically examining and conferring with a patient, comprising: providing an examination table having a seat and a back in articulating interconnection, said seat having an upper surface for receiving and supporting a lower portion of a patient and said back having a front surface for supporting an upper portion of the patient, and further having a roll of sanitary cover sheets containing medical illustrations of portions of a human body relevant to medical conditions of a patient maintained on a rear surface of said back, said dispenser selectively dispensing said cover sheets with medical illustrations over at least one of said front surface of said back and upper surface of said seat; positioning a patient alongside the physician adjacent to the examination table and medical illustrations; and with reference to said medical illustrations, engaging a dialogue between the physician and patient regarding an assessment of a medical issue of the patient.

### DESCRIPTION OF DRAWINGS

For a complete understanding of the various aspects, structures, and method of the invention, reference should be made to the following detailed description and accompanying drawings, wherein:

FIG. 1 is an illustration of a reclining examination table with a roll of separable and dispensable cover sheets having medical illustrations thereon according to the invention;

FIG. 2 represents medical illustrations of the interrelationship of the heart with the pulmonary and systemic circuits of the human body as may be implemented on the examination table cover sheet of the invention;

FIG. 3 is a medical illustration of the lungs of the human body as may be implemented on examination table cover sheets according to the invention;

FIG. 4 is a grouping of medical illustrations of the human heart as may be implemented on cover sheets for examination tables according to the invention;

FIG. 5 is grouping of medical illustrations of upper extremities such as the shoulder, arm and elbow of a human being, as may be implemented on a cover sheet of an examination table according to the invention;

FIG. 6 presents a grouping of illustrations of lower extremities of the human body as might be implemented on cover sheets of an examination table according to the invention; and

FIG. 7 is a preferred dimensional illustration of a cover sheet for an examination table made in accordance with the invention and showing medical illustrations on the section of the cover sheet that would cover the back of the examination table.

### DETAILED DESCRIPTION OF PREFERRED EMBODIMENTS

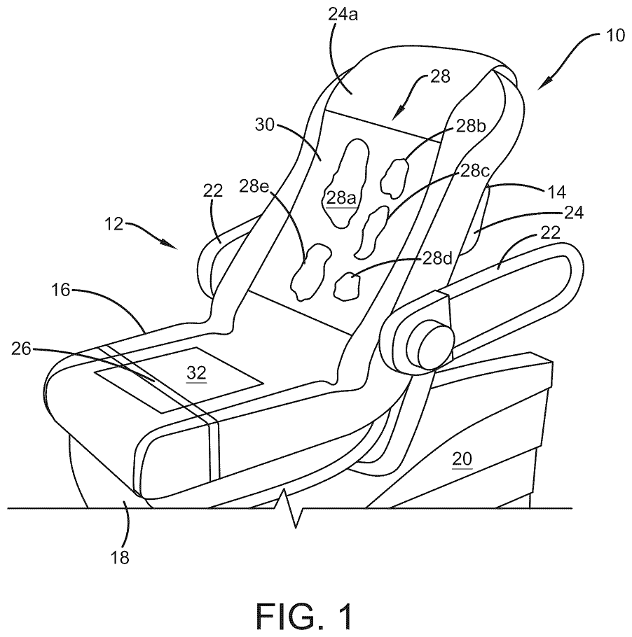

Referring now to the drawings, and more particularly FIG. 1, a patient examination and education system made in accordance with the invention as designed generally by the numeral 10. The system includes a patient examination table 12, having a back 14, seat 16, and leg rest 18. Such examination tables are well known in the art and, for most, the back 14, seat 16 and leg rest 18 articulate with respect to each other as examination table 12 may be operative as a chair, a bed or a reclined chair as the needs might require.

A base 20 is provided to receive and maintain the examination table 12 and to house the mechanism for articulation of the component parts thereof. Hand rails or bed rails 22 may be positioned along each side of the back 14 to prevent the patient from rolling off of the examination table 12 when in its reclined position.

It has previously been known that a roll of cover sheets 24 may be maintained on the back of the seat back 14, as shown. Typically, the roll 24 is a continuous section of sheet that passes over the top of seat back 14, downward along the front of the back 14, across the seat 16, and to the leg rest 18. A sheet retaining bar 26 may traverse the seat 16, receiving and retaining the sheet thereunder as it passes from the roll 24. For purposes of the invention herein, the sheet retaining bar 26 is transparent.

As shown in FIG. 1, the sheet portion 24a dispensed from the roll 24 and retained under the retaining bar 26, is populated by one or more medical illustrations 28 presented on the portion 30 of the cover sheet covering the back 14 of the examination table 12. As shown in FIG. 1, five illustrations 28a-28e are employed in a particular instance, there being more or less depending on the medical practice involved. While only the back 14 is shown as having medical illustrations on the cover sheet covering the back, it will be appreciated that similar illustrations may be presented on a seat section 32 of the cover sheet as shown. The invention herein contemplates such medical illustrations both on the back portion and seat portion of the cover sheet. It will be appreciated that the back portion, being somewhat vertical, is easier to view than the seat portion, which is horizontal. Either or both may contain medical illustrations on the cover sheet.

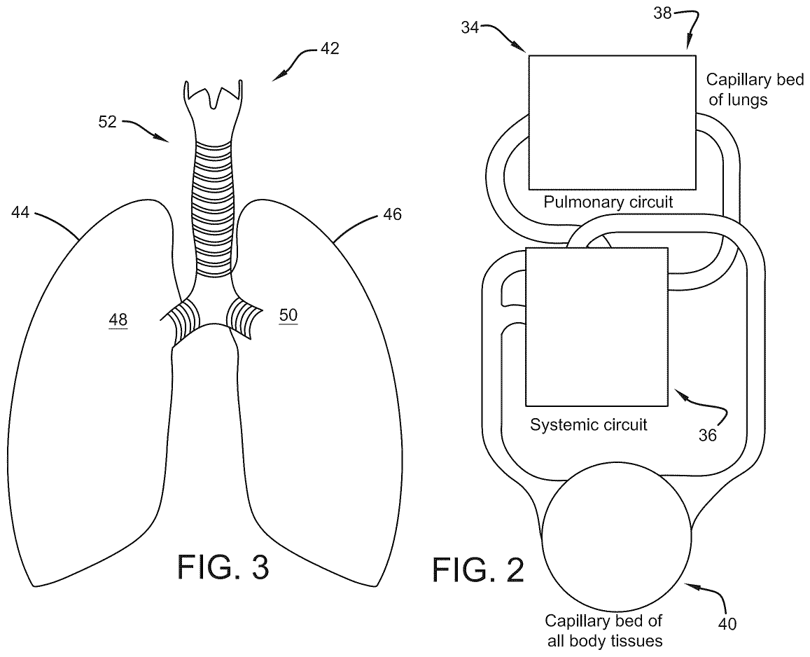

It will be appreciated that the invention contemplates the implementation of any of numerous medical illustrations to be presented on the cover sheet sections 24a of the roll 24, the particular illustrations being dependent upon the nature of the practice of the physicians employing the particular examination table. The orientations and details of the medical illustrations may be selected to accommodate the needs of the particular medical practice. Such medical illustrations are shown generally herein, the desired or necessary specificity being readily appreciated by those skilled in the art. As shown in FIG. 2, the medical illustration 34 is that of the interrelationship of the heart with the pulmonary and systemic circuits of the human body and may be positioned at the regions of 28, 32, as shown in FIG. 1. The medical illustration 34 of FIG. 2 shows the heart 36, lungs 38 and capillary bed of the body tissues 40 of a human being, accommodating an appreciation and understanding of the interoperations of the heart, lungs and the capillary bed for body tissues where gas exchange occurs. It is preferred that colors may be employed, such as red, showing oxygen-rich blood, and blue, showing oxygen poor or carbon dioxide rich blood. The various elements of the illustration are appropriately labeled to accommodate discussion and understanding between physician and patient or other participants. Not only are the elements of the components of the circuits labeled, but their function is explained in text. The medium of the cover sheet is not only sanitary, but given to the imparting of handwritten notes or texts, as by pencil, pen or marker. The illustration of FIG. 2 would find particular utility on a cover sheet for a pulmonary physician or cardiologist.

Referring to FIG. 3, it can be seen that a medical illustration of a pulmonary region suitable for presentation on a sheet cover for an examination table is designated generally by the numeral 42. As illustrated, the illustration presents the right lung 44, left lung 46, right bronchus 48, and left bronchus 50. Also presented for discussion purposes are the superior, middle and inferior lobar bronchus for both the right and left lung along with the associated intermediate bronchus. Also presented are the various cartilages and ligaments 52. These elements are labeled for discussion and large enough to accommodate highlighting and notes added by the physician or patient. Such an illustration would be suitable for use in a pulmonary physician examining room and contain such illustrative detail as recognized by physicians as necessary for medical discussions with patients.

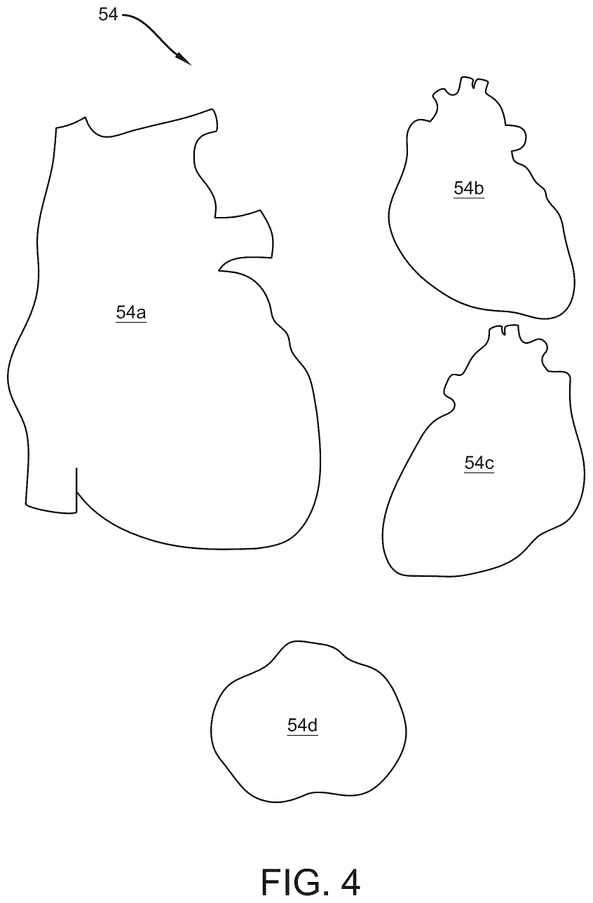

With reference to FIG. 4, a grouping of medical illustrations of the human heart are presented together as may be received on the back section 30 or seat section 32 of an examining table cover sheet 24a. For use in the examining room of a cardiologist, various anterior, posterior, and sectional views 54a-54d of the heart and related valves may be shown, with appropriate detail, labeling of elements and features and with room for notes, highlights, questions and the like.

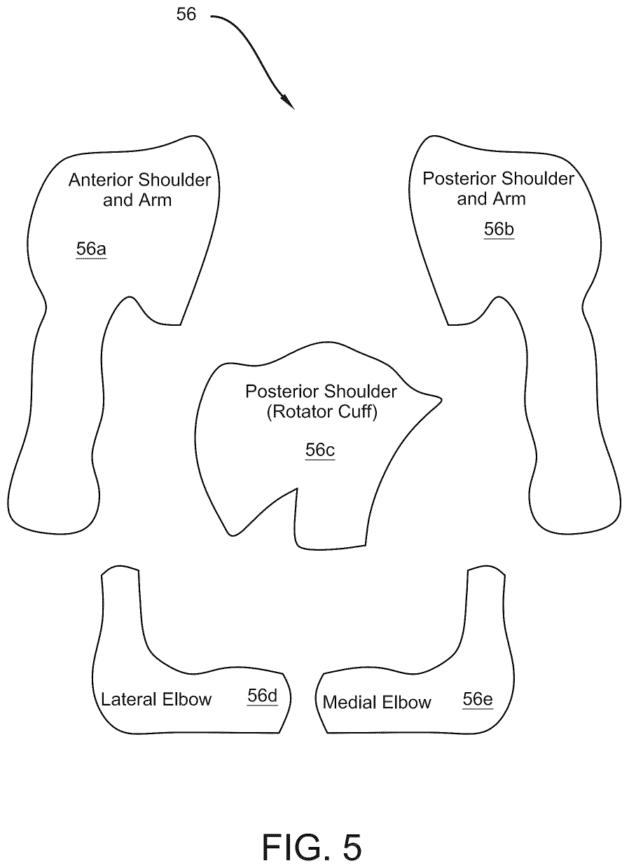

In an orthopedic surgeon's examining room, a grouping of medical illustrations 56, as shown in FIG. 5, may be appropriately presented on the cover sheet 24a of an examination table 12. Presented in FIG. 5 are detailed illustrations of the upper extremities including, by way of example, those of the anterior shoulder and arm 56a, posterior shoulder and arm 56b, posterior shoulder (rotator cuff) 56c, lateral elbow 56d, and medial elbow 56e, all showing combinations of skeletal structure, organs, muscles, joints, ligaments, tendons, cartilage and the like. Again, the notable elements and features are identified and named and room is left for entry of highlighting and notes.

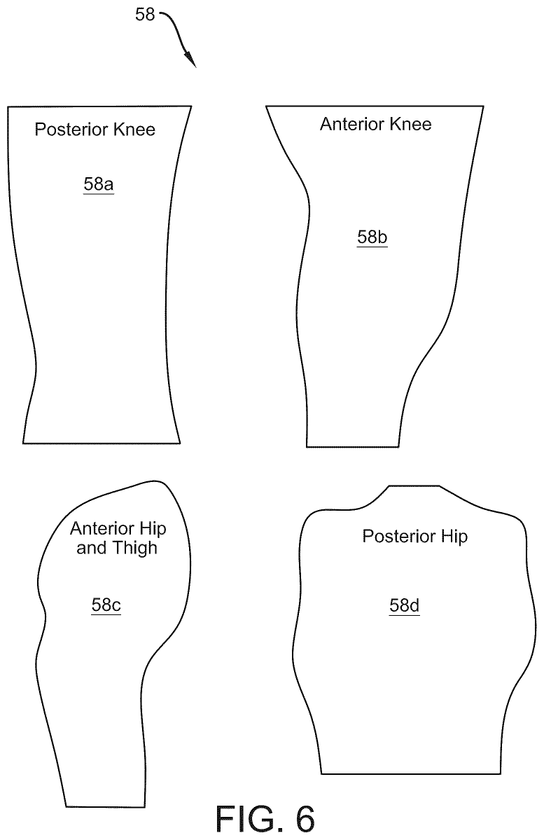

FIG. 6 presents a compilation of medical illustrations of lower extremities 58 that might also be used on the cover sheets for the examination table of an orthopedic surgeon. Here, sample lower extremities include a posterior knee 58a, anterior knee 58b, anterior hip and thigh 58c, posterior hip 58d and the like. Again, the elements and features are identified with sufficient space for entry of notes and highlighting.

It will be appreciated that any of numerous medical illustrations may be employed for presentation on the examination table cover sheets. The medical illustrations employed will most commonly be those associated with the specific practice of the physician or treating medical personnel that are involved. The human body accommodates an extensive number of such illustrations. The invention contemplates customized cover sheet arrangements with the illustrations taken from a library of medical illustrations or generated independently and originally to satisfy the needs of specific medical practices.

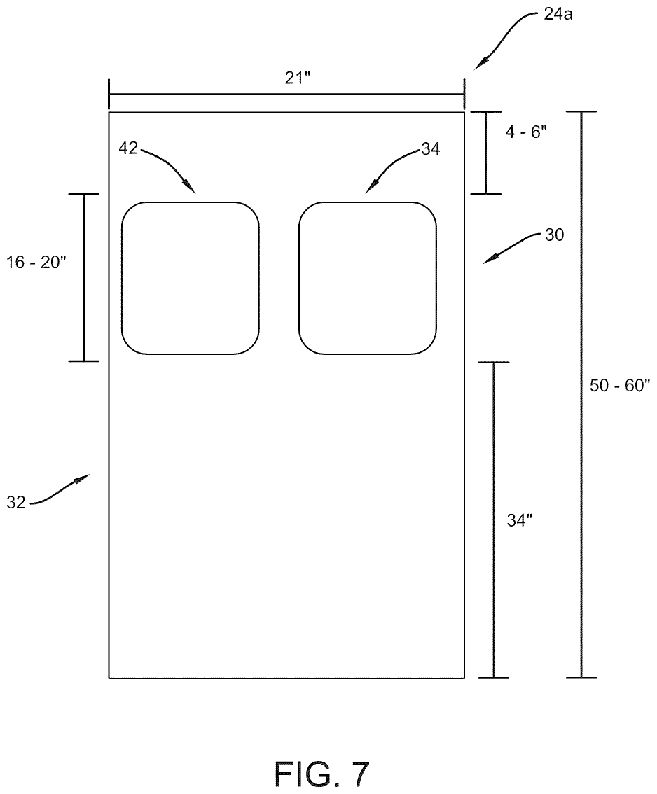

FIG. 7 shows a cover sheet section 24a as presented in FIG. 1 covering the top surfaces of the examination table 12. It will be appreciated that the cover sheet section 24a originates as a continuum of the cover sheet paper of the roll 24 maintained on the back side of the back 14 of the examination table 12. The cover sheet sections 24a may be separated by transverse perforation lines of separation for ease of separating a sheet 24a for replacement by a fresh sheet. Alternatively, the roll may be without perforations, and simply be torn as against the sheet retaining bar 26 to position a clean sheet in its place.

The cover sheet section 24a of FIG. 7 shows the preferred dimensions of the cover sheet section to accommodate coverage for a standard patient examination table 12. The illustration of FIG. 7 shows the medical illustrations 34, 42 of FIGS. 2 and 3 positioned in the back section area 30, it being understood that other illustrations may be provided in the seat section 32. It will be appreciated that the dimensions shown allow for the presentation of illustrations that are easily viewed, highlighted and written upon as the need might be.

Given the foregoing, it will be understood that visual illustrations truly help bridge the gap between physician communication and patient understanding, especially given the complexity of healthcare today. Currently, patient examination table cover sheets are found in nearly every outpatient medical office and are primarily used for keeping the examination table clean between patients. However, the invention herein changes the interaction between physician and patient, leading to enhanced efficacy of outpatient-physician visits. With examination table cover sheets having specialty-specific pre-printed detailed images, physicians will be able to educate the patient regarding health issues, planned procedures and future management strategies, all using visualization to guide the conversation. Such provides not only a stimulus for the healthcare provider to use visual illustrations to guide the discussion/education for the patient, but also provides a scaffold for aiding in the education of the patient and understanding health and management issues with visual cues. Such is a very effective way to bridge the knowledge gap between the physician and the patient. The examination cover sheets of the invention will (1) aid in patient understanding, (2) allow physicians to have more efficient visits, and (3) continue to provide sterilization between subsequent patient visits.

The cover sheet section 24a, dimensioned as in FIG. 7, is large enough to allow for appropriate physician-patient interaction, providing enough space to allow for hand drawing, notes, and the like while explaining to the patient matters concerning health conditions and planned procedures. In that regard, the seat section 32 may be left fully blank without medical illustration to allow for recordations made during the physician-patient discussions.

As presented above, the examination table cover sheets are specialty oriented across a broad range of medical practices. For example, physicians specializing in certain orthopedic procedures, such as knee replacements, will be provided with specific anatomy illustrations showing the knee joint in multiple views to help guide discussions, including (1) pathology of the knee and (2) specific regions of the knee where the surgery will occur. This allows the patient to visually see the extent of the disease of the knee and what surgery will be performed. This is in comparison to the antiquated way of communication such as the use of verbal medical jargon, resulting in patients leaving visits confused as to what exact surgery they are getting and why. The assistance these cover sheets can render to various health providers is somewhat limitless. A pulmonologist may show their patients where their lung cancer is, while a cardiologist may show the patient where a new coronary artery stent has been or will be placed, and a breast surgeon may show where the patient's breast mass is and the anticipated surgery. The invention presents not only examination table cover sheets with medical illustrations, but a change in the methodology in medical practice, patient-physician interaction and patient education/experience. Outpatient office experience for everyone, physicians and patients alike, benefit.

The invention just described not only prompts physicians to educate the patients about their health, injuries, or future procedures/surgeries, but also reminds patients to actually prompt the physicians to use the sheets to educate them. Patients, seeing these education sheets, will be reminded to ask physicians more detailed questions about things they may not have understood during the appointment.

The examination table cover sheets of the invention actually change the dynamics of a medical examination room. Presently, such rooms are dull, void of imagery, and rather antiquated. The advent of an examination table with cover sheets as those presented herein add professional imagery to the room and also allow patients to educate themselves and prompt questions while waiting for the physician. The patient examination and education system 10 of the invention accommodates physician personalization. The medical illustration on the examination table cover sheets are largely dependent on the physician using the examination sheets. Cardiologists will have various cardiac images to help guide medical discussions with patients, ranging from basic cardiac and valvular structures to coronary arterial anatomy. This allows cardiologists to have a broad range of discussions with their patients, from management of heart failure to where they need a stent to manage their coronary artery disease. Similarly, orthopedic surgeons are able to have various joint images, including shoulder, knee, hip and the like. This again allows such surgeons to have various images to help guide medical conversations about injuries, potential surgeries and the like. This is a marked difference from current practice of medicine in which physicians use verbal dialogue alone for explaining surgeries or injuries, often leaving patients unsettled or confused about their health or future procedures. The customization can be very specific. Physicians can personalize the sheet based upon their own specialties. For example, if an orthopedic surgeon specializes in shoulder interventions, the medical illustrations on the cover sheet sections 24a may be personalized to that physician's practice and portray only shoulder anatomies.

The patient examination and education system 10 of the invention improves both the patients'and physicians'experiences tremendously. The system helps prompt the physician to start medical discussions and guide education with the patient, while making the appointment more efficient by aiding in targeted image-guided dialogue mitigating the need to draw pictures and diagrams themselves.

Thus it can be seen that the various aspects of the invention have been satisfied by the structures and techniques presented above. While in accordance with the patent statutes only the best mode and preferred embodiments of the invention have been presented in detail, the invention is not limited thereto or thereby. Accordingly, for an appreciation of the true scope and breadth of the invention, reference should be made to the following claims.

What is claimed is:

1. A patient examination and education system, comprising:
an examination table having a seat and a back in articulating interconnection, said seat having an upper surface for receiving and supporting a lower portion of a patient and said back having a front surface for supporting an upper portion of the patient; and
a dispenser of information relevant to medical conditions of patients maintained on a rear surface of said back, said dispenser selectively dispensing said information over at least one of said front surface of said back and upper surface of said seat.

2. The patient examination and education system according to claim 1, wherein said dispenser of information comprises a roll of paper.

3. The patient examination and education system according to claim 2, wherein said paper comprises sanitary cover sheets for covering said seat and back of said examination table.

4. The patient examination and education system according to claim 3, wherein said information relevant to medical conditions of patients is carried by said sanitary cover sheets.

5. The patient examination and education system according to claim 4, wherein said information relevant to medical conditions of patients comprises medical illustrations of portions of the human body.

6. The patient examination and education system according to claim 5, wherein said sanitary cover sheets are given to the receipt of handwritten notes with areas adjacent said illustrations for receipt of such handwritten notes.

7. The patient examination and education system according to claim 6, wherein said cover sheets with medical illustrations and handwritten notes are separable from said roll to be retained by the patient.

8. The patient examination and education system according to claim 5, wherein said medical illustrations are tailored to a medical practice of a physician, surgeon, or other practitioner or specialist.

9. The patient examination and education system according to claim 5, wherein said medical illustrations are illustrations of skeletal structure, organs, muscles, joints, tendons, and cartilage.

10. A method of a physician medically examining and conferring with a patient, comprising:
providing an examination table having a seat and a back in articulating interconnection, said seat having an upper surface for receiving and supporting a lower portion of a patient and said back having a front surface for supporting an upper portion of the patient, and further having a roll of sanitary cover sheets containing medical illustrations of portions of a human body relevant to medical conditions of a patient maintained on a rear surface of said back, said dispenser selectively dispensing said cover sheets with medical illustrations over at least one of said front surface of said back and upper surface of said seat;
positioning a patient alongside the physician adjacent to the examination table and medical illustrations; and
with reference to said medical illustrations, engaging a dialogue between the physician and patient regarding an assessment of a medical issue of the patient.

11. The method of a physician medically examining and conferring with a patient according to claim 10, wherein the physician and patient enter handwritten notes and highlighting on the sanitary cover sheets, in areas adjacent said medical illustrations.

12. The method of a physician medically examining and conferring with a patient according to claim 11, wherein said cover sheets with medical illustrations and handwritten notes are separated from said roll and retained by the patient.

13. The method of a physician medically examining and conferring with a patient according to claim 11, wherein a conversation between physician and patient is encouraged and guided by the medical illustrations on the cover sheets.
